(19) United States
(12) Patent Application Publication
(10) Pub. No.: US 2003/0084482 A1
(43) Pub. Date: May 1, 2003

(54) Production of proteins in plants

(76) Inventors: Gerald Hall, Morrisville, PA (US); Newell Bascomb, Brookside, NJ (US); Mark Bossie, Robbinsville, NJ (US)

Correspondence Address: EDWARDS & ANGELL, LLP., P.O. BOX 9169, BOSTON, MA, 02209, US

(21) Appl. No.: 10/165,420

(22) Filed: Jun. 7, 2002

Related U.S. Application Data
(60) Provisional application No. 60/297,103, filed on Jun. 8, 2001.

(51) Int. Cl. A01H 5/00; C07H 21/04; C12N 9/00; C12N 15/82; C12N 5/04

(52) U.S. Cl. 800/288; 435/69.1; 435/183; 435/320.1; 435/419; 536/23.2

(57) ABSTRACT

The present invention provides compositions and methods for producing proteins in plants, particularly proteins that in their native state require the coordinate expression of a plurality of structural genes in order to become biologically active. The ultimate products typically possess therapeutic, diagnostic or industrial utility.

### RELATED APPLICATION

This Application claims priority under 35 U.S.C. § 119(e) to U.S. Provisional Application No. 60/297,103, filed Jun. 8, 2001, the entirety of which is incorporated by reference herein.

### FIELD OF THE INVENTION

This invention is related to the production in plants of antibodies and other complex proteins.

### BACKGROUND OF THE INVENTION

Recombinant DNA technology entails the modification of the genetic make-up of an organism with a specific segment of DNA for some beneficial purpose. This has led to the engineering of microbes, cell cultures, plants and animals to produce valuable products for a wide variety of applications. An important consideration for doing this is the ability to produce the product of interest in the most cost effective manner than what could have previously been accomplished by standard methods. In essence, genetic engineering has expanded the portfolio of products that can now be produced through the most favorable and cost effective production systems.

While initially this work was performed in bacterial systems, it is now routine to transform many types of organisms including various microbial eukaryotes (yeast and other fungi), plant and animal cells in culture and to produce transgenic whole plants and animals. There are numerous challenges to face in the production of products through any transgenic approach. While microbial systems often offer advantages up-front in speed of cloning and producing transformed cells, there are often difficulties in the scale-up from laboratory to large fermentation vessels. In addition, while bacteria efficiently synthesize and secrete recombinant proteins and enzymes they do not generally have the machinery to perform all of the required post-translational modifications. Some fungi are able to produce secreted glycoproteins; however, the type of glycans and processing are different from that seen in animal systems.

Mammalian and insect cell cultures have become widely used for the production of a variety of proteins, with probably the most significant advantage being post-translation processing. Otherwise, the media, equipment and fastidious culture conditions drive up production cost and are a distinct disadvantage to these systems. Similar to the case with microbial cultures, scale-up also becomes a significant issue because translation from lab-scale to large-scale is often not direct. Yet another disadvantage of such systems is the potential for harboring virions or prions of concern to human health.

Transgenic animals have also been described for producing human proteins in milk, excreted in the urine or produced via eggs of avian species. In general, there is still the potential problem of animal viruses and disease causing organisms. Additionally, scale-up and maintenance costs of the production population (herd) can be significant and very time consuming. Like animal cell culture, transgenic animals should provide proteins with the requisite post-translation modifications.

Using plants as a recombinant protein expression system or “bioreactor” has been discussed as an attractive alternative to bacterial, yeast, insect, animal and cell-based production systems. There are many benefits to producing proteins in plants and the use of plants for the production of transgenic proteins is gaining widespread support.

Plant production systems allow for ease of purification free from animal pathogenic contaminants. Transformation methods exist for a large number of plant species. In the case of many seed plants and agricultural crops, the methods and infrastructure already exist for harvesting and handling large quantities of material. Scale-up is relatively straightforward and is based simply on production of seed and planting area. Thus, there is a substantial reduction in the cost of goods, reduced risks of mammalian viral or prion contamination, and relatively low capital requirements for raw material and production facilities as compared to producing similar material via mammalian cell culture or transgenic animals.

Plants generally suffer only a single significant drawback and that is in the area of post-translational glycosylation of proteins. However, it has been demonstrated that in many cases the alternative carbohydrate modifications of plants do not cause deleterious effects or undesirable immunogenic properties to the glycoprotein.

A number of production systems have been developed for expressing proteins in plants. These include expressing protein on oil bodies (Rooijen, et al. Plant Physiology 109:1353-1361 (1995); Liu, et al. Molecular Breeding 3:463-470(1997)), through rhizosecretion (Borisjuk, et al. Nature Biotechnology 17:466-469 (1999)), in seed (Hood, et al. Molecular Breeding 3:291-306 (1997); Hood, et al. In Chemicals via Higher Plant Bioengineering [edited by Shahidi, et al.] Plenum Publishing Corp. pp. 127-148 (1999); Kusnadi, et al. Biotechnology and Bioengineering 56:473-484 (1997); Kusnadi, et al. Biotechnology and Bioengineering 60:44-52 (1998); Kusnadi, et al. Biotechnology Progress 14:149-155 (1998); Witcher, et al. Molecular Breeding 4:301-312 (1998)), as epitopes on the surface of a virus (Verch, et al. Journal of Immunological Methods 220:69-75 (1998); Brennan, et al., Journal of Virology 73:930-938 (1999); Brennan, et al., Microbiology 145:211-220 (1999)), and stable expression of proteins in potato tubers (Arakawa, et al. Transgenic Research 6:403-413 (1997); Arakawa, et al. Nature Biotechnology 16:292-297 (1998); Tacket, et al., Nature Medicine 4:607-609 (1998)). Recombinant proteins can also be targeted to seeds, chloroplasts or to extracellular spaces to identify the location that gives the highest level of protein accumulation.

It is generally accepted that the basic functional segment of DNA coding for a product includes a promoter followed by a protein-coding region and then a terminator. This basic, single cistronic (also termed “monocistonic”) format has long been the standard for expressing genes in any organism. According to the ribosome-scanning model, traditional for most eukaryotic mRNAs, the 40S ribosomal subunit binds to the 5′-cap and moves along the non-translated 5′-sequence until it reaches an AUG codon (Kozak, Adv. Virus Res. 31:229-292 (1986); Kozak, J. Mol. Biol. 108:229-241 (1989)). Although for the majority of eukaryotic mRNAs only the first open reading frame (ORF) is translationally active, there are different mechanisms by which mRNA may function polycistronically (Kozak, Adv. Virus Res. 31:229-292 (1986)) such that a plurality of coding regions are expressed without each one being controlled by a separate promoter.

Patent publication WO98/54342 teaches methods for the simultaneous expression of desired genes in plants using internal ribosome entry sites (IRES) derived from plant viruses. The publication also discloses that tobamovirus IRES elements provide an internal translational pathway for 3′-proximal gene expression from bicistronic chimeric RNA transcripts in plant, animal, human and yeast cells, and that foreign genes can be inserted downstream from the IRES and expressed. Patent publication WO 00/789085 describes using the IRES elements in gene constructs designed to permit stacking of multiple crop protection traits in a crop (i.e., herbicide resistance and expression of an insecticidal toxin, Bt) or to express genes that can alter a plant's metabolites, causing it to produce polyhydroxyalconates (PHA's) which serve as precursors to certain types of plastics.

### SUMMARY OF THE INVENTION

The present invention provides compositions and methods for producing proteins in plants, particularly proteins that in their native state require the coordinate expression of a plurality of structural genes in order to become biologically active. The ultimate products typically possess therapeutic, diagnostic or industrial utility.

Accordingly, one aspect of the present invention is directed to a recombinant nucleic acid molecule, or expression unit, containing from 5′ to 3′, a transcription initiator and a plurality of structural genes, each separated by an internal ribosome binding sequence (IRES). In preferred embodiments, the transcription initiator is a promoter functional in a plant cell (although is not necessarily naturally found in a plant). The transcription initiator may additionally comprise enhancer sequences or other regulatory elements for modulating the degree of expression and/or specificity of expression (e.g., providing temporal and/or spatial regulation of transcription).

Preferably, the structural genes encode subunits of a multi-subunit protein. As used herein, a “multi-subunit protein” is a protein containing more than one separate polypeptide or protein chain associated with each other to form a single globular protein, where at least two of the separate polypeptides are encoded by different genes. In one preferred aspect, a multi-subunit protein comprises at least the immunologically active portion of an antibody and is thus capable of specifically combining with an antigen. For example, the multi-subunit protein can comprise the heavy and light chains of an antibody molecule or portions thereof. Multiple antigen combining portions can be encoded by different structural genes to generate multivalent antibodies.

However, any multi-subunit protein is encompassed within the scope of the present invention. Exemplary multisubunit proteins include, but are not limited to, heterodimeric or heteromultimeric proteins, such as T Cell Receptors, MHC molecules, proteins of the immunoglobulin superfamily, nucleic acid binding proteins (e.g., replication factors, transcription factors, etc), enzymes, abzymes, receptors (particularly soluble receptors), growth factors, cell membrane proteins, differentiation factors, hemoglobin like proteins, multimeric kinases, and the like.

In another aspect, the structural genes encode the components of protein complexes which function coordinately, e.g., such as enzyme complexes, complexes of differentiation factors, replication complexes, and the like.

In one aspect, the invention provides a first expression unit comprising a transcription initiator functional in a plant cell, a structural gene encoding one subunit of a first multi-subunit protein (e.g., comprising the heavy or light chains of an antibody molecule) and a first reporter gene encoding a selectable marker active in plant cells. A second expression unit also may be provided which contains a transcription initiator functional in the plant cell, one or more structural genes which encode another subunit of a second multi-subunit protein (such as the heavy or light chain of an antibody molecule) and a second reporter gene encoding a selectable marker different from that in the first expression unit and which is also active in plant cells. One or more expression units can comprise origins of replication, prokaryotic and or eukaryotic. Multiple different types of eukaryotic origins may be provided for example, to allow replication of the expression unit(s) in one or more of: plant cells, mammalian cells, yeast cells, insect cells, and the like.

In other preferred embodiments, the structural genes of an expression unit encode one or more proteins required to process an immature protein into a mature biologically active form. For example, the structural gene may encode a protease required to process an immature protein, such as preproinsulin, into a mature form, insulin, by cleaving the protein. Genes encoding the immature protein may be provided as part of the same expression unit or as part of a different expression unit.

In yet other preferred embodiments, the recombinant nucleic acid molecule or expression unit contains 5′ to at least one structural gene, a sequence encoding a targeting peptide sequence (e.g., transit peptide) for directing the expression product(s) of the gene(s) to certain locations in or outside the plant cell. In one aspect, each structural gene comprises a 5′ targeting sequence for directing the structural genes to selected locations. The 5′ targeting sequences may be the same or different, e.g., certain combinations of gene products may be targeted to the same or different locations. The recombinant nucleic acid molecule may further comprise a selectable marker gene and/or a polyadenylation sequence. Preferably, the polyadenylation sequence is the 3′-most portion of the expression unit.

Another aspect of the present invention is directed to a method for producing proteins in plants, comprising: preparing a vector comprising the recombinant nucleic acid molecule; introducing the vector into the plant cell, thus producing a transformed plant cell; and selecting for plants derived from the transformed plant cell that express the plurality of coding sequences. In preferred embodiments, the expression products are targeted to a specific location such as the cell membrane, extracellular space or a cell organelle, e.g., a plastid such as a chloroplast. In other preferred embodiments, the plant cell is an Arabidopsis cell. The transformed plant cells, transgenic plants containing the recombinant nucleic acid molecules, including plants regenerated from the transformed plant cells, plant parts, and seed derived from the transgenic plants, are also provided.

The present invention provides genetic constructs that are useful for either transient or stable expression in plants and plant cells and result in expression of active biomolecules not endogenously produced by a plant.

### BRIEF DESCRIPTION OF THE DRAWINGS

The objects and features of the invention can be better understood with reference to the following detailed description and accompanying drawings.

FIG. 1 is a schematic representation of a nucleic acid construct of the present invention;

FIG. 2 is a schematic representation of a nucleic acid construct of the present invention;

FIG. 3 shows the sequence of the chloroplast targeting peptide from ribulose 1,5-bisphosphate carboxylase small subunit (GenBank ACCESSION X02353);

FIG. 4 presents a sequence comparison of the amino terminal portion of the plant calreticulin protein aligned with the amino terminal region of various antibody genes;

FIG. 5 is a plasmid map of pICP1176;

FIG. 6 is a plasmid map of pICP1221;

FIG. 8 is a plasmid map of pICGHpolyAb1;

FIG. 7 is a plasmid map of pICP1177; and

FIG. 9 is a plasmid map of pICGHpolyAb4.

FIG. 10 is a plasmid map of pXB1500.

FIGS. 11A and 11B are schematic representations of nucleic acid constructs of the present invention useful in producing insulin.

### DETAILED DESCRIPTION OF THE INVENTION

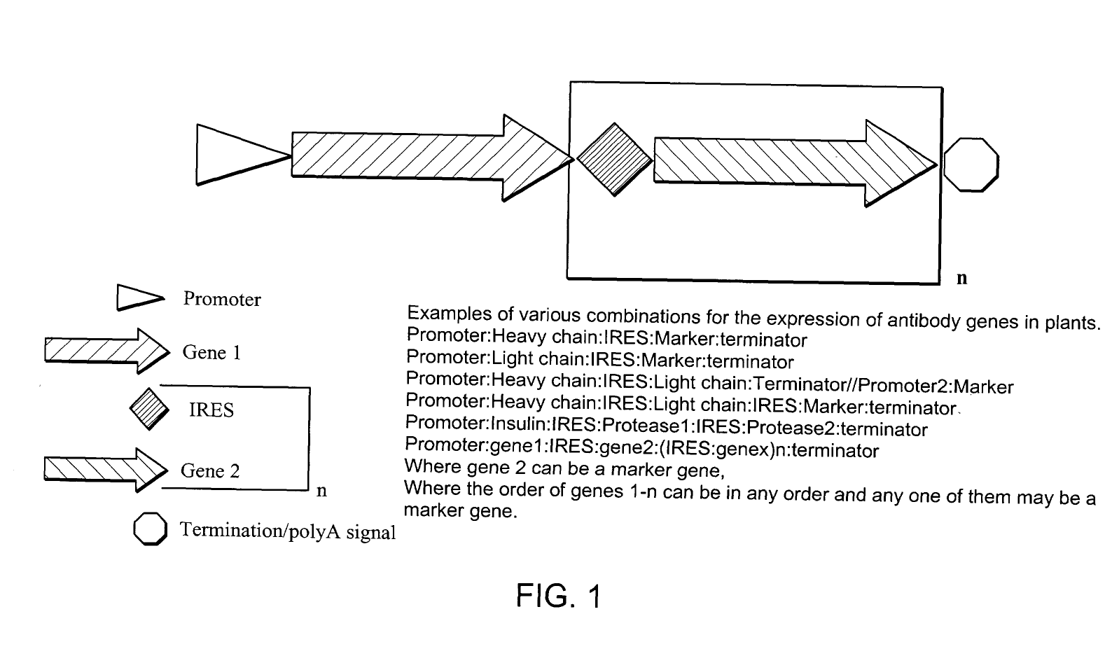

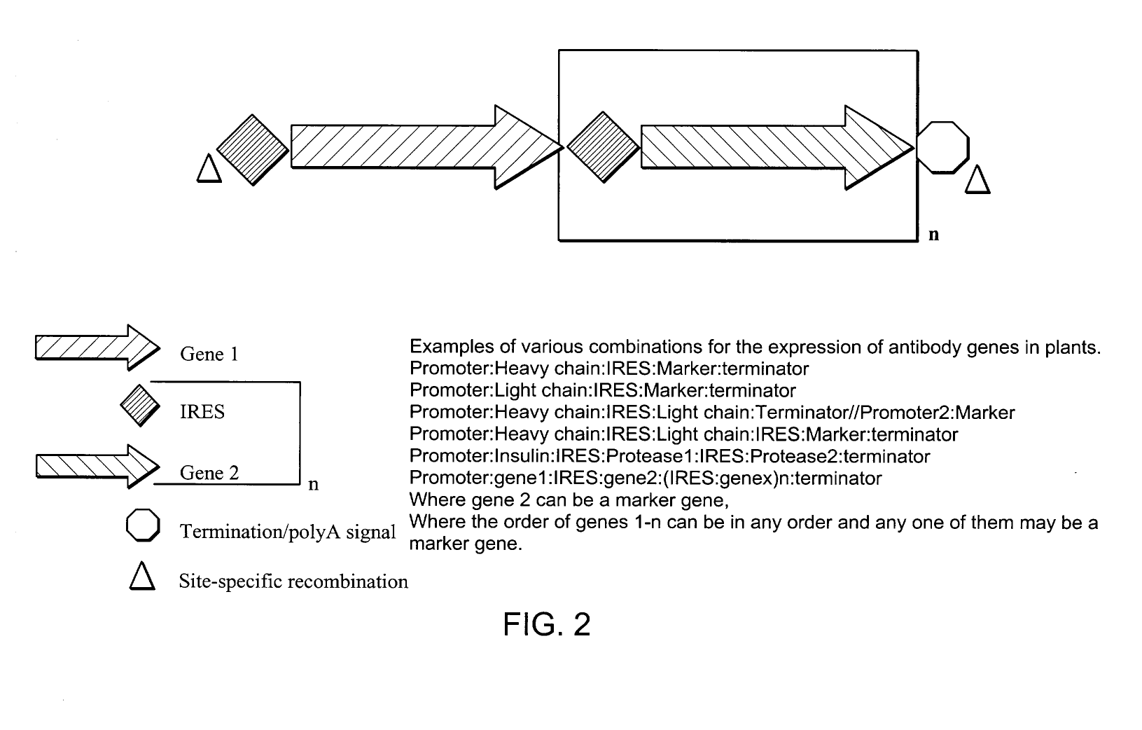

Various genetic constructs in accordance with the present invention are schematically illustrated in FIGS. 1 and 2. FIG. 1 illustrates a construct in which a promoter drives the first gene in a series of genes, each of which is separated by an IRES element. The IRES sequence initiates cap-independent translation in the selected plant cell. In preferred embodiments, a polyadenylation signal is inserted immediately 3′ to the sequence of the last gene to be expressed to allow for efficient processing of the transcript. Transcription of the constructs results in formation of one polycistronic mRNA. Ribosomes bind independently at the 5′ end of the RNA as well as at each IRES element allowing independent but coordinate expression of all proteins in the polycistronic mRNA.

FIG. 2 illustrates another embodiment of the present invention wherein an IRES element is positioned at the 5′ end of the DNA construct rather than a promoter. This enables the genes on the construct to be expressed in a manner that is regulated by the transcriptional activity of the host locus into which the DNA construct inserts during transformation. In a related embodiment, the DNA construct contains sequences that permit site-specific integration into a previously defined chromosomal locus having a desirable transcriptional expression profile. Thus, in embodiments represented by FIG. 2, the 5′ IRES element enables the genes to be expressed based on the transcriptional control of the genetic locus into which the gene construct has inserted.

Plant Promoters

The promoter may be constitutive, tissue-specific, developmentally regulated or otherwise inducible or repressible, provided that it is functional in the plant cell. A large number of plant promoters have been described which are capable of directing gene expression that is either constitutive, or in some fashion regulated. Regulation may be based on temporal, spatial or developmental cues, environmentally signaled, or controllable by means of chemical inducers or repressors and such agents may be of natural or synthetic origin and the promoters may be of natural origin or engineered. Transcription initiation regions may comprise promoters and one or more additional regulatory elements, such as enhancers. Promoters also can be chimeric, i.e., derived using sequence elements from two or more different natural or synthetic promoters.

Plant promoters can be selected to control the expression of transgenes in different plant tissues by methods are known to those skilled in the art (Gasser & Fraley, Science 244:1293-99 (1989)). The cauliflower mosaic virus 35S promoter (CaMV) and enhanced derivatives of CaMv promoter (Odell et al., Nature, 3(13):810 (1985)), actin promoter (McElroy et al., Plant Cell 2:163-71 (1990)), AdhI promoter (Fromm et al., Bio/Technology 8:833-39 (1990), Kyozuka et al., Mol. Gen. Genet. 228:4048 (1991)), ubiquitin promoters, the Figwort mosaic virus promoter, mannopine synthase promoter, nopaline synthase promoter and octopine synthase promoter and derivatives thereof are considered constitutive promoters. Regulated promoters are described as light inducible (e.g., small subunit of ribulose biphosphatecarboxylase promoters), heat shock promoters, nitrate and other chemically inducible promoters (see, for example, U.S. Pat. Nos. 5,364,780; 5,364,780; and 5,777,200).

Tissue specific promoters are used when there is reason to express a protein in a particular part of the plant. Leaf specific promoters may include the C4PPDK promoter preceded by the 35S enhancer (Sheen, 15 EMBO, 12:3497-505 (1993)) or any other promoter that is specific for expression in the leaf. For expressing proteins in seed, the napin gene promoter (U.S. Pat. Nos. 5,420,034 and 5,608,152), the acetyl-CoA carboxylase promoter (U.S. Pat. Nos. 5,420,034 and 5,608,152), 2S albumin promoter, seed storage protein promoter, phaseolin promoter (Slightom et. al., Proc. Natl. Acad Sci. USA 80:1897-1901 (1983)), oleosin promoter (Plant et al., Plant Mol. Bio. 25:193-205 (1994); Rowley et. al., 1997, Biochim. Biophys. Acta. 1345:1-4 (1997); U.S. Pat. No. 5,650,554; PCT WO 93/20216), zein promoter, glutelin promoter, starch synthase promoter, and starch branching enzyme promoter are all useful.

IRES Elements in Plants

The IRES element may be one of those previously described (Atebekov et al. WO 98/54342 and U.S. Pat. No. 6,376,745; Snell, WO-A 2000078985) or an artificial IRES active in plant cells (i.e., a synthetic or engineered IRES). For multi-IRES-containing constructs, it may be useful to use IRES elements having different DNA sequences. Recently a new tobamovirus, crTMV, has been isolated from Oleracia officinalis L. plants and the crTMV genome has been sequenced (6312 nucleotides) (Dorokhov et al. Doklady of Russian Academy of Sciences 332:518-522 (1993); Dorokhov et al. FEBS Lett. 350:5-8 (1994)).

Unlike the RNA of typical tobamoviruses, translation of the 3 ′-proximal CP gene of crTMV RNA occurs in vitro and in planta by a mechanism of internal ribosome entry which is mediated by a specific sequence element, IRES_{CP} (Ivanov et al. Virology 232, 32-43 (1997)). The results indicated that the 148-nucleotide region upstream of the CP gene of crTMV RNA contained IRES_{CP} promoting internal initiation of translation in vitro and in vivo (protoplasts and transgenic plants).

Recently it has been shown (Skulachev et al., Virology 263:139-154 (1999)) that the genomic RNAs of tobamoviruses contain a sequence upstream of the MP gene that is able to promote expression of the 3′-proximal genes from chimeric mRNAs operably linked to the sequence in a cap-independent manner in vitro. The 228-nucleotide sequence upstream from the MP gene of crTMV RNA (IRES_{MP228}^{CR}) mediates translation of the 3′-proximal GUS gene from bicistronic transcripts. A 75-nnucleotide region upstream of the MP gene of crTMV RNA is still as efficient as the 228-nucleotide sequence. Therefore the 75-nucleotide sequence contains an IRES_{MP} element (IRES_{MP75}^{CR}). It has been found that in similarity to crTMV RNA, the 75-nucleotide sequence upstream of genomic RNA of a type member of tobamovirus group (TMV UI) also contains IRES_{MP75}^{UI} element capable of mediating cap-independent translation of 3′-proximal genes.

The tobamoviruses provides a new example of internal initiation of translation, which is markedly distinct from IRES's shown for picornaviruses and other viral and eukaryotic mRNAs. The IRES_{MP} element capable of mediating cap-independent translation is contained not only in crTMV RNA but also in the genome of a type member of tobamovirus group, TMV UI, and another tobamovirus, cucumber green mottle mosaic virus. Consequently, different members of tobamovirus group contain IRES_{MP}.

By way of example, two specific IRES elements are used in demonstration of this invention. Nucleotide sequences of two IRES's from the genome of the crucifer tobacco mosaic virus (crTMV):

| | |
|---|---|
| IRESmp75^{cr}: | |
| 5′TTCGTTTGCTTTTTGTAGTATAATTAAATATTTG | (SEQ ID NO. 1) |
| TCAGATAAGAGATTGTTTAGAGATTTGTTCTTTGTT | |
| TGATA3′ | |
| IREScp148^{cr}: | |
| 5′GAATTCGTCGATTCGGTTGCAGCATTTAAAGCGG | (SEQ ID NO. 2) |
| TTGACAACTTTAAAAGAAGGAAAAAGAAGGTTGAAG | |
| AAAAGGGTGTAGTAAGTAAGTATAAGTACAGACCGG | |
| AGAAGTACGCCGGTCCTGATTCGTTTAATTTGAAAG | |
| AAGAAA3′ | |

Proteins Encoded By Structural Genes

In one aspect, the proteins encoded by the expression units and expressed in methods of the present invention are those that in their native state require the coordinate expression of a plurality of structural genes in order to become biologically active. In one case, the protein requires the assembly of a plurality of subunits to become active. In another case, the protein is produced in immature form and requires processing, e.g., proteolytic cleavage by one or more additional proteins or protein modification (e.g., phosphorylation, glycosylation, prenylation, ribosylation, etc) to become active.

Non-limiting examples described in the demonstration of this invention are antibodies (e.g., monoclonal antibodies) and insulin. In both classes of proteins, the present invention demonstrates not only the ability to produce the functional molecules by a method of coordinate expression but also that the genetic constructs and subsequent polycistronic mRNA's disclosed herein, while not normal in plant cells, are properly recognized by the protein secretion apparatus of the cell. Notably, monoclonal antibodies may be produced by the constructs and methods of the invention without the need to generate hybridoma cells.

The genes for monoclonal antibodies can be obtained from murine, human or other animal sources. Alternatively, they can be synthetic, e.g., chimeric or modified forms of the genes encoding the heavy chain or light chain components of an antibody molecule. The order of the coding regions, e.g., heavy and light, or light then heavy, is not important. Genes coding for Heavy and Light polypeptides (e.g., such as variable heavy and variable light polypeptides) can be derived from cells producing IgA, IgD, IgE, IgG or IgM. Methods for preparing fragments of genomic DNA from which immunoglobulin variable region genes can be cloned are well known in the art. See for example, Herrmann et al., Methods in Enzymol., 152:180-183 (1987); Frischauf, Methods in Enzymol., 152:183-190 (1987); Frischauf, Methods in Enzymol., 152:199-212 (1987).

Probes useful for isolating the genes coding for immunoglobulin products include the sequences coding for the constant portion of the V H and V L sequences coding for the framework regions of V H and V L and probes for the constant region of the entire rearranged immunoglobulin gene, these sequences being obtainable from available sources. See, for example, Early and Hood, Genetic Engineering, Setlow and Hollaender eds., Vol. 3:157-188, Plenum Publishing Corporation, New York (1981); and Kabat et al., Sequences of Immunological Interests, National Institutes of Health, Bethesda, Md. (1987).

Insulin is an example of a protein that, in its native environment, is encoded and translated in a precursor form and then modified by one or more proteolytic cleavage steps to form the mature and functional form of the protein. Following translation, processing of the preproinsulin protein to a mature form includes proteolytic cleavage steps including removal of the amino terminal secretion signal sequence (a common step in the eukaryotic secretion pathway) and processing at internal sites by a subtilisin family protease, such as PC2 and PC 1/PC3 proteases, and trimming by carboxypeptidase E. Cleavage results in the release of an internal peptide, the C-peptide and A and B peptides. The A and B peptides undergo intra and inter-chain disulfide bond formation to form the mature insulin protein.

As the cellular compartments of the eukaryotic secretion pathway provide a preferred environment for proper protein maturation, folding and disulfide bond formation, expressing human or animal proteins in this manner in plants will likewise prove advantageous for the production of properly formed mature proteins. Other methods of synthesizing mature insulin involve separately expressing each of the A and B peptides and then providing a suitable reducing environment in vitro to bring about disulfide bond formation (U.S. Pat. Nos. 4,421,685 and 4,559,300).

Numerous types of polycistronic constructs can be prepared to produce insulin in accordance with the present invention. In one embodiment, a polycistronic gene construct contains the insulin-coding region along with its own secretion signal or a plant secretion signal, as well as structural genes encoding the proteolytic processing enzymes. The gene for human insulin (GenBank Accession J00265) can be cloned using a variety of methods known to those skilled in the art. A preferred form of the clone is a cDNA derived from the mature mRNA thus eliminating the intron sequences and reducing the overall size of the cloned gene. Similarly, the genes encoding the proteolytic enzymes (PC2, PC1 /PC3 and carboxypeptidase E can all be cloned using known DNA sequence information, e.g., comprising one or more of the sequences below in one or more expression units as described above.

| Structural Gene | GenBank Description |
|---|---|
| Human insulin | DEFINITION Human insulin gene, |
| | complete cds. |
| | ACCESSION J00265 (GenBank) |
| PC2 proprotein converting | DEFINITION Homo sapiensproprotein |
| enzyme | convertase subtilisin/kexin type 2 |
| | (PCSK2), mRNA. |
| | ACCESSION XM_012963 (GenBank) |
| PC3 (PC1) proprotein converting | DEFINITION Homo sapiensproprotein |
| enzyme | convertase subtilisin/kexin type 1 |
| | (PCSK1), mRNA. |
| | ACCESSION XM_003674 |
| CPE carboxypeptidase E enzyme | DEFINITION Homo sapiens |
| | carboxypeptidase E (CPE), mRNA. |
| | ACCESSION XM_003479 (GenBank) |

In each of these cases the preferred form of the genes is the cDNA derived from mature mRNA or its equivalent DNA sequence. One may generate numerous polycistronic vectors to bring about the expression of all of these components in the necessary proportions to achieve a high level of expression of mature insulin within the plant. Thus, the invention provides for the complete synthesis in a plant of a processed mature therapeutic protein by combining all of the necessary genes into polycistronic vectors.

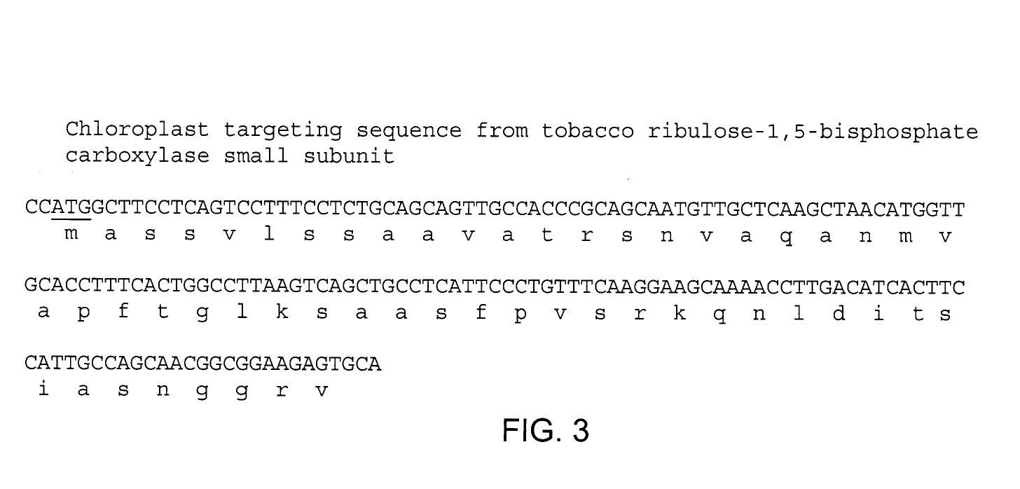

In a preferred embodiment, the nucleic acid construct or expression unit comprises, from 5′ to 3′, a promoter driving expression of the human insulin gene followed by an IRES (preferably cp148 or mp75), the coding region for CP2, a second IRES, the coding region for CP3, a third IRES and the coding region for CPE. The entire segment is then terminated at the 3′ end with a proper plant transcription termination and polyadenylation signals to ensure most efficient processing of the transcript. See FIG. 3A. Although a single order of the genes is described, the most optimal order of the coding regions for any given sequence of coding regions for a therapeutic protein may be determined in accordance with standard techniques and expression units having different orders of genes are encompassed within the scope of the invention.

In other embodiments, the constructs and methods of the present invention may be modified in such a way that the structural gene encoding the immature form of insulin is introduced into the plant cell separately, e.g., after the introduction of the construct containing the structural genes encoding processing protein(s). Thus, a “host” processing plant is prepared and may be propagated until the expression unit comprising the insulin gene in introduced. In the case of insulin for example, the polycistronic gene construct would not contain the insulin coding region and the promoter would drive expression of the first (PC2) processing enzyme followed by IRES's driving expression of the PC3 and CPE genes. The insulin gene is then introduced into a plant as either a stable genetic element or by methods for transient expression. Schematic representations of such constructs are shown in FIG. 3B. The products of each of these genes are localized to the appropriate sub-cellular compartments most resembling the process as it occurs in human cells.

Targeting Sequences

When proteins are synthesized in a cell they can be targeted to specific sub-cellular or extracellular locations by virtue of targeting sequences. In some cases the sequence of amino acids is synthesized as the amino terminal portion of the polypeptide and is cleaved by proteases after or during the translocation or localization process. For instance, the model of the protein secretion pathway in eukaryotes is that following ribosome binding to mRNA and initiation of translation the nascent polypeptide chain emerges. If it is a protein destined for secretion, the emerging amino terminus of the protein is recognized by signal recognition particle (SRP)that bring about a temporary stalling of translation while the mRNA, ribosome and SRP complex docks with the endoplasmic reticulum (ER). After docking, translation resumes, although now the polypeptide chain is co-translationally translocated through to the ER lumen. It is possible that proteins be translocated post-translationally; however, this process in vivo is far less efficient and generally is not considered the normal route of entry into the ER.

U.S. Pat. No. 5,474,925 describes an expression construct utilizing a signal peptide translationally fused to a recombinant protein which targets the protein to the cellulose matrix of the cell wall. This enables the isolation of the protein along with the recoverable cellulose matrix and is particularly useful for expressing proteins in cotton plants. Thus, in one embodiment of the invention, the expression unit may comprise a structural gene fused in frame to a sequence encoding such a signal peptide.

In another aspect, proteins may be targeted to the interstitial fluids of a plant permitting a protein, such as an antibody, preferably, a monoclonal antibody, to be isolated directly from the interstitial fluids. One exemplary way of isolating proteins from interstitial fluids is described in U.S. Pat. No. 6,284,875. Thus, in one embodiment the expression unit may comprise a structural gene fused in frame to a targeting sequence from a protein secreted into interstitial fluids. Such proteins are described in U.S. Pat. No. 6,284,875, for example.

In the present invention, and particularly in preferred embodiments, e.g., wherein the structural genes encode the heavy and light chains of an antibody molecule, the structural genes include targeting peptides for directing the expression product to a secretory pathway. As antibodies are normally secreted proteins—the secretion process plays an important role in the production of the mature antibody molecules. To accomplish this in plants, the genes are synthesized (e.g., cloned) having either their native mammalian signal peptide encoding region, or as a fusion in which a plant secretion signal peptide is substituted. The fusion between the signal peptide and the protein should be such that upon processing by the plant, the resultant amino terminus of the protein is identical to that which is generated in the human host.

Targeting proteins to the endomembrane system of a plant is a preferred embodiment of the present invention as it provides for the proper maturation of the amino terminus of the protein. Further localization to specific regions of the endomembrane system can be accomplished if the protein of interest either has or is engineered to contain additional targeting information.

Targeting to organelles such as plastids (e.g., chloroplast) and mitochondria is also advantageous for achieving the desired amino-terminal maturation as targeting to either of these locations is dictated by an amino-terminal signal sequence that subsequently undergoes a cleavage event. In preferred embodiments, the signaling peptides direct the expression products to a plastid (e.g., a chloroplast) or other subcellular organelle. An example is the transit peptide of the small subunit of the alfalfa ribulose-biphosphate carboxylase (Khoudi, et al., Gene 197:343-5 (1997)). A peroxisomal targeting sequence refers to any peptide sequence, either N-terminal, internal, or C-terminal, that can target a protein to the peroxisomes, such as the plant C-terminal targeting tripeptide SKL (Banjoko, et al. Plant Physiol. 107:1201-08 (1995)).

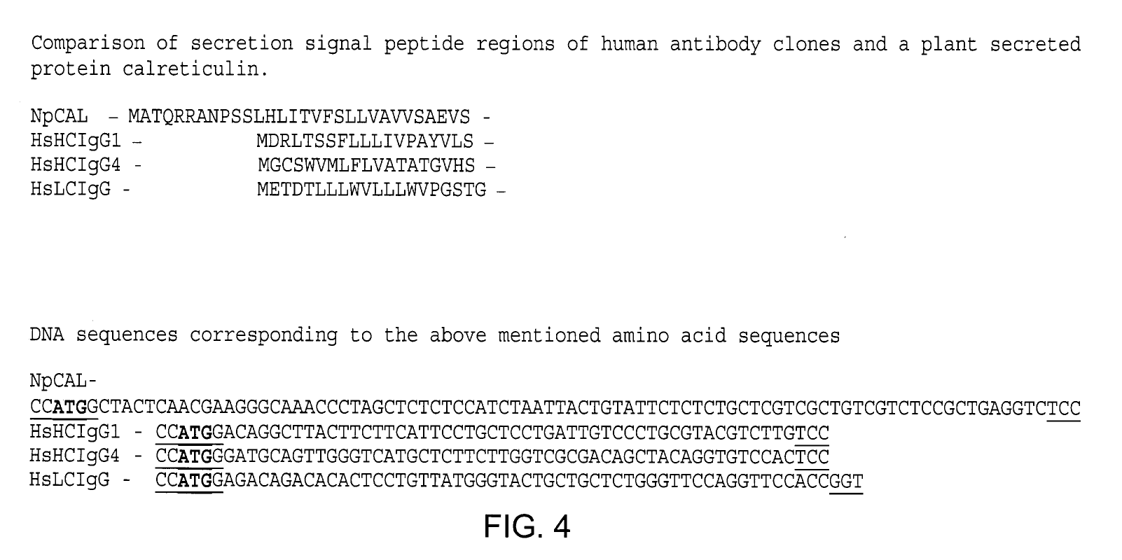

On the other hand, nuclear localization signals are not naturally restricted to the 5′ end position (amino terminus) and are not proteolytically removed by any known cellular mechanisms. FIG. 4 shows the sequence of the chloroplast targeting peptide from the tobacco nuclear gene encoding ribulose 1,5-bisphosphate carboxylase small subunit (GenBank ACCESSION X02353). Upon entry, the signaling or transit peptide is removed by the action of an organellular protease. A gene fusion comprising this sequence at the 5′ end, to the sequence beginning at the first amino acid of the mature form of the protein of interest (i.e., the antibody heavy or light chain) is useful in producing the mature form of the protein.

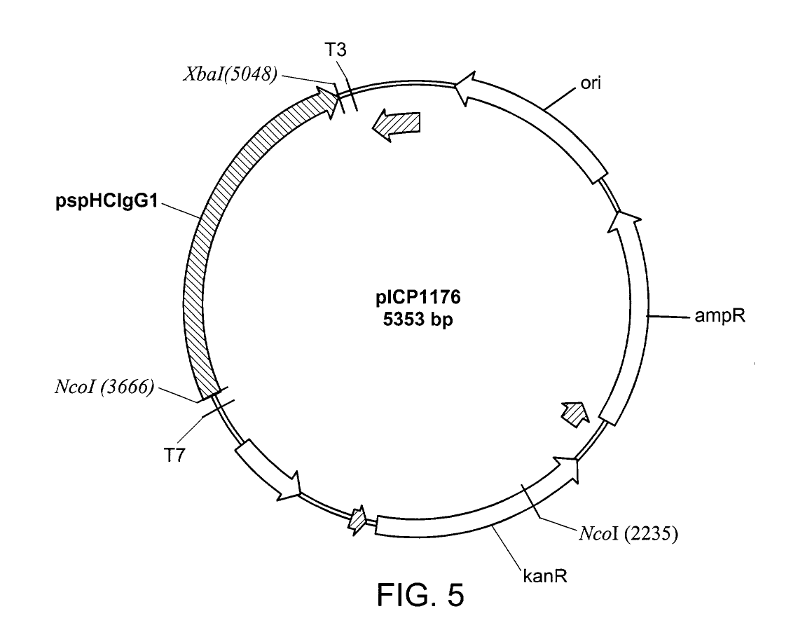

The signal sequences for targeting proteins to the endomembrane system for localization in the vacuole or for secretion are similar in plants and animals. FIG. 5 shows a sequence comparison of the amino terminal portion of the plant calreticulin protein aligned with the amino terminal region of a few antibody genes. The alignment includes that portion of the antibody proteins which is made as part of the pre-protein but is not present in the final mature protein following processing through the secretory pathway. It is not untypical for such signal sequences to vary somewhat in length as is seen in this example where the plant signal peptide is 10-11 amino acids longer than the mammalian sequences, they all clearly share common features known to be associated with eukaryotic secretion signal peptides. Signaling peptides may be adapted for use in the present invention (e.g., prepared with suitable ends for cloning in-frame with any other gene) in accordance with standard techniques.

Fusion Proteins

Structural genes may also encode fusion proteins. For example, a structural gene encoding a polypeptide subunit of a multimeric or multi-subunit protein or of a protein to be processed may comprise a sequence encoding an effector polypeptide. As used herein, an “effector molecule” refers to an amino acid sequence such as a protein, polypeptide or peptide and can include, but is not limited to, regulatory factors, enzymes, antibodies, toxins, and the like. Non-limiting examples of desired effects produced by an effector molecule, include, inducing cell proliferation or cell death, to initiate an immune response or to act as a detection molecule for diagnostic purposes (e.g., the fusion may encode a fluorescent polypeptide such as GFP, EGFP, BFP, YFP, EBFP, and the like).

Selectable Markers and/or Reporter Genes

Selectable markers, such as antibiotic (e.g., kanamycin and hygromycin) resistance, herbicide (glufosinate, imidazlinone or glyphosate) resistance genes or physiological markers (visible or biochemical) encoded by reporter genes are used to select cells transformed with the nucleic acid constructs of the invention. Non-transgenic cells (i.e., non-transformants) on the other hand, are either killed or preferentially do not grow under the selective conditions. Reporter genes may be included in the construct or they may be contained in the vector that ultimately transports the construct into the plant cell. As used herein, a “reporter gene” is any gene which can provide a cell in which it is expressed with an observable or measurable phenotype.

Preferably, expression of reporter genes yields a detectable result, e.g., providing a visual calorimetric, fluorescent, luminescent or biochemically assayable product; and/or a selectable marker, allowing for selection of transformants based on physiological responses (e.g., a growth differential, change in proliferation rate, state of differentiation, and the like). Expression of a reporter gene in a cell can cause the cell to display a visual physiologic or biochemical trait. Commonly used reporter genes include lacZ (β-galactosidase), GUS (β-glucuronidase), GFP (green fluorescent protein), luciferase, or CAT (chloramphenicol acetyltransferase), which are easily visualized or assayable. Such genes may be used in combination with or instead of selectable markers to enable one to easily pick out clones of interest. Selectable markers can also include molecules that facilitate isolation of cells which express the markers. For example, a selectable marker can encode an antigen which can be recognized by an antibody and used to isolate a transformed cell by affinity-based purification techniques or by flow cytometry. Reporter genes also may comprise sequences which are detected by virtue of being foreign to a plant cell (e.g., detectable by PCR, for example). In this embodiment, the reporter need not express a protein or cause a visible change in phenotype.

Plant Transformation Methods for transferring and integrating a DNA molecule into the plant host genome are well known. Methods such as Arabidopsis vacuum-infiltration or dipping are preferred because many plants can be transformed in a small space, yielding a large amount of seed to screen for transformants. Agrobacterium typically transfers a linear DNA fragment (T-DNA) with defined ends (T-DNA borders) making it a preferred method as well. Direct DNA transformation, such as microinjection, chemical treatment, or microprojectile bombardment (biolistics) are also useful. Barring any limitations on the size of the recombinant DNA construct, polycistronic gene encoding sequences according to the invention can be delivered into plants using viral vectors. The plant cells transformed may be in the form of protoplasts, cell culture, callus tissue, suspension culture, leaf, pollen or meristem.

The transformed cells may then in suitable cases be regenerated into whole plants in which the new nuclear material is stably incorporated into the genome. Both transformed monocotyledonous and dicotyledonous plant may be obtained in this way. There are a variety of plant types that can be transformed with the nucleic acid constructs of the present invention. Examples of other genetically modified plants which may be produced include field crops, cereals, fruit and vegetables such as canola, tobacco, sugarbeet, cotton, soya, maize, wheat, barley, rice, sorghum, tomatoes, mangoes, peaches, apples, pears, strawberries, bananas, melons, potatoes, carrot, lettuce, cabbage, onion. Preferred plants are Arabidopsis, Brassica species, maize, alfalfa, soybean, tobacco, crucifera, cottonseed, sunflower and legumes.

Isolation of Proteins

After cultivation, the transgenic plant is harvested to recover the produced multi-subunited protein or processed protein (and/or other proteins produced by structural genes according to the invention). This harvesting step may comprise harvesting the entire plant, or only the leaves, or roots or cells of the plant. This step may either kill the plant or, if only the portion of the transgenic plant is harvested, may allow the remainder of the plant to continue to grow.

After harvesting, protein isolation may be performed using methods routine in the art. For example, at least a portion of the plant may be homogenized, and the protein extracted and further purified. Extraction may comprise soaking or immersing the homogenate in a suitable solvent. As discussed above, proteins may also be isolated from interstitial fluids of plants, for example, by vacuum infiltration methods, as described in U.S. Pat. No. 6,284,875.

Purification methods include, but are not limited to, immuno-affinity purification and purification procedures based on the specific size of a protein/protein complex, electrophoretic mobility, biological activity, and/or net charge of the multimeric protein to be isolated.

### EXAMPLES

The present invention will now be described by way of several working examples. These examples are for purposes of illustration and are not meant to limit the invention in any way.

### Example 1

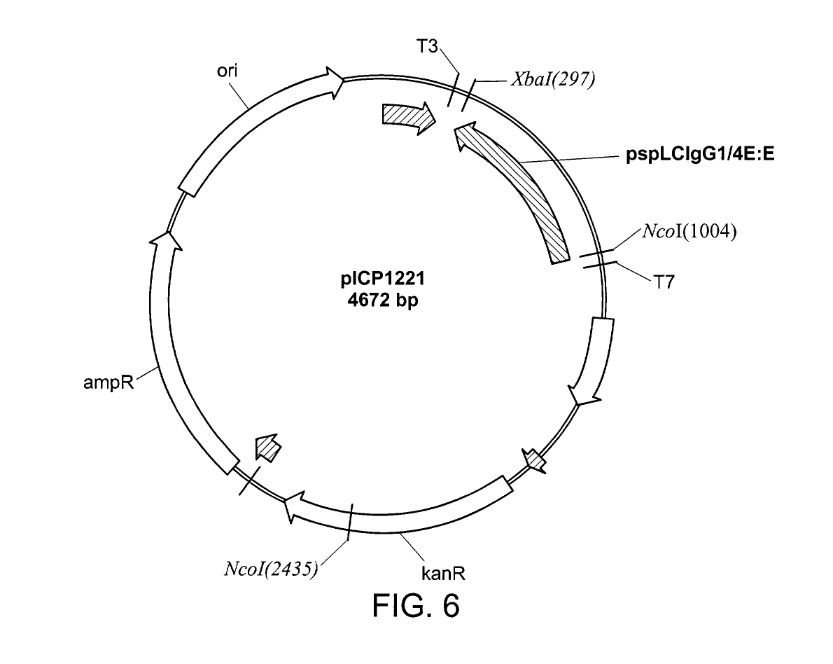

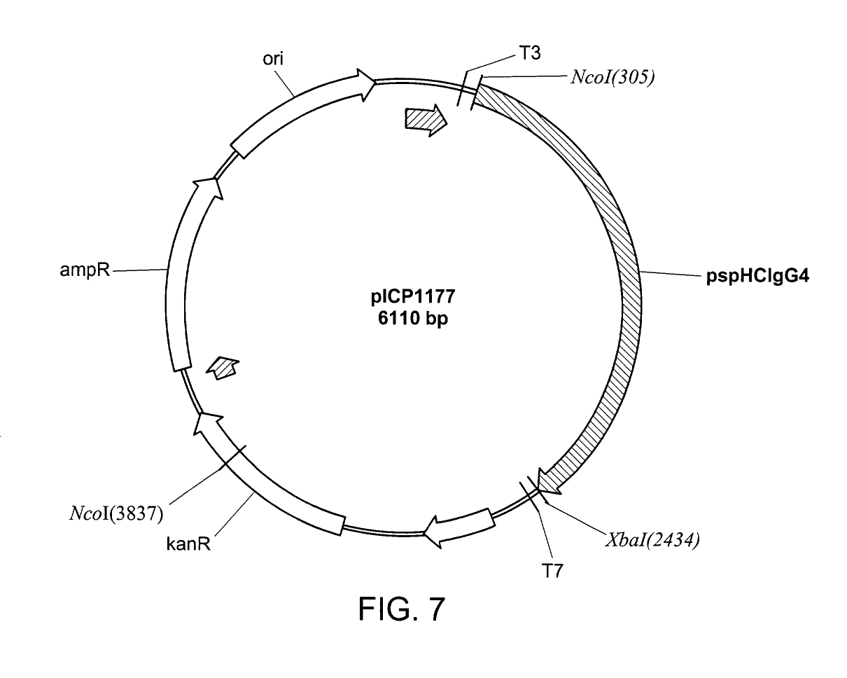

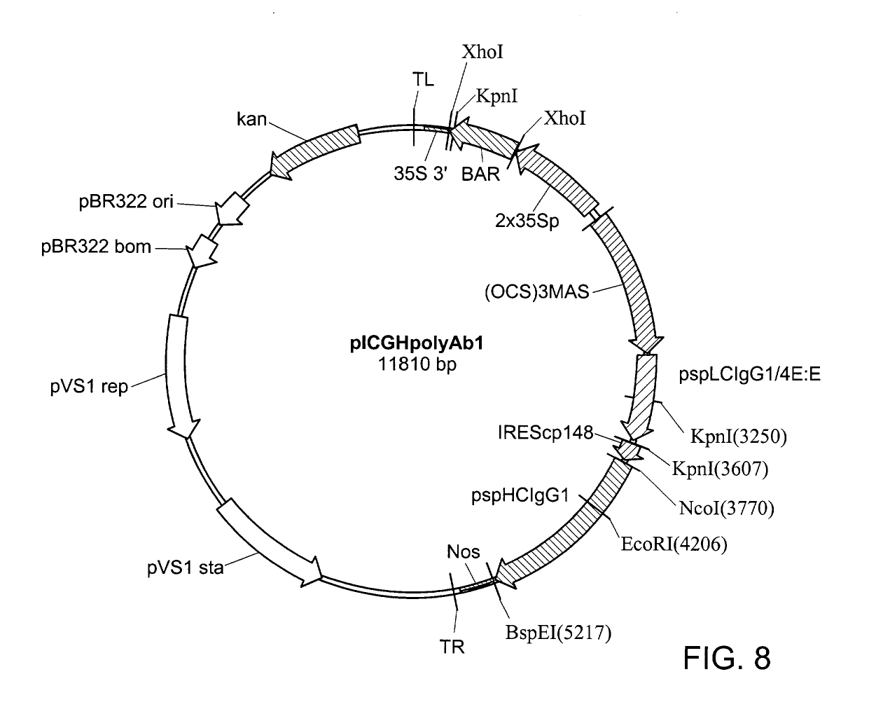

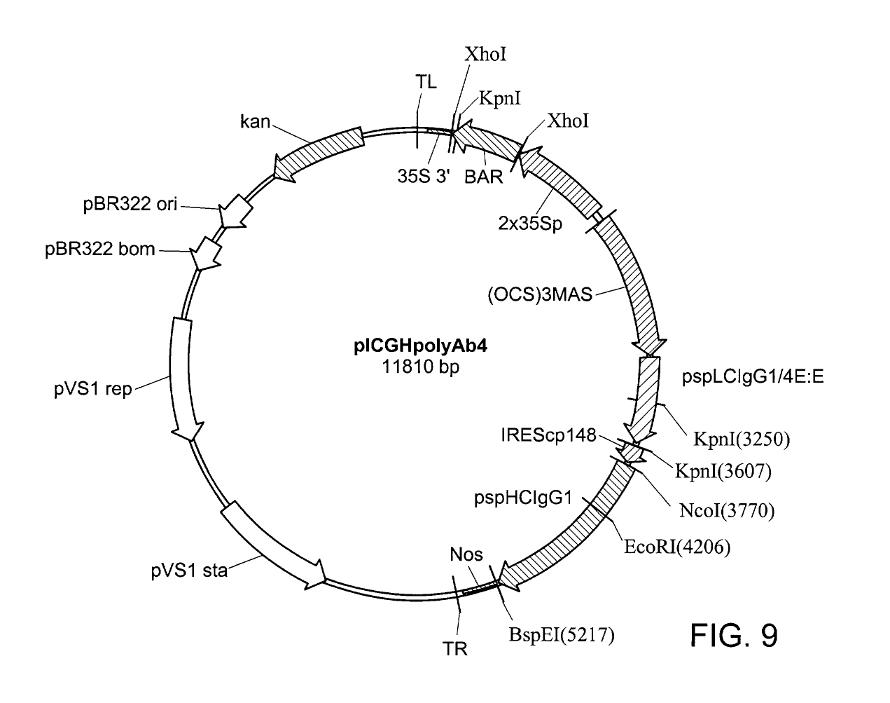

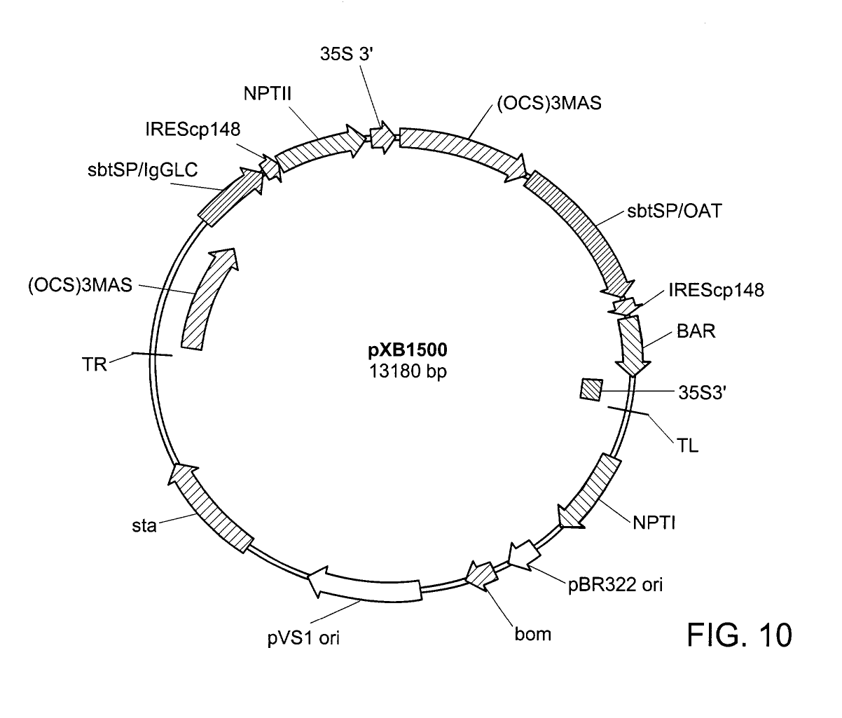

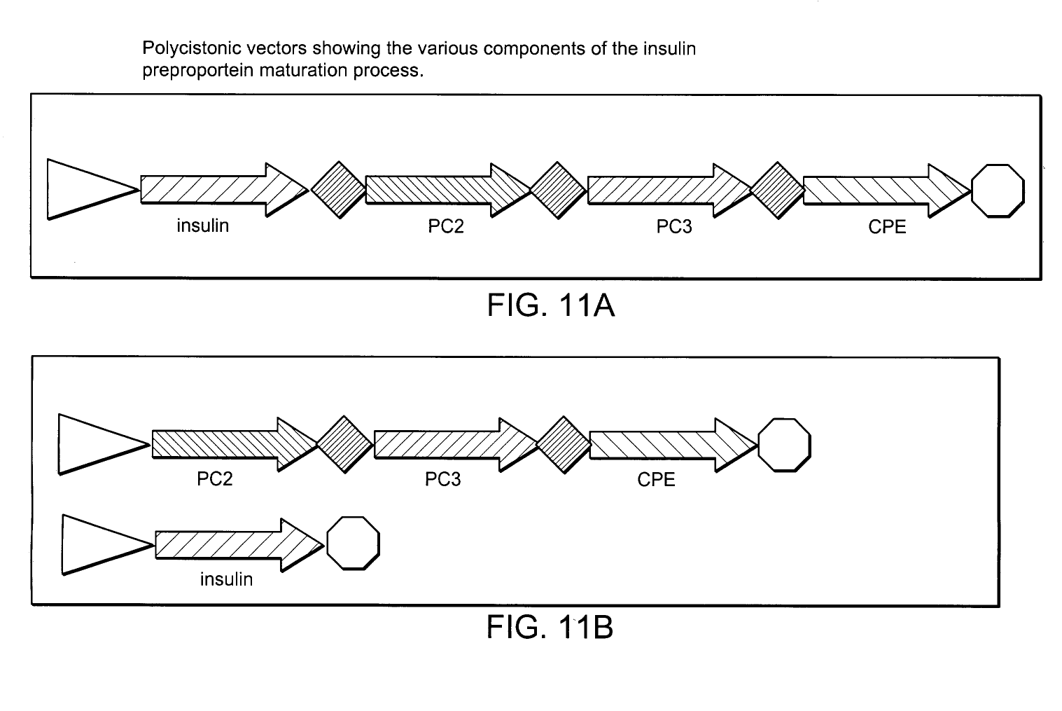

Plasmid ICP1176 (FIG. 6) includes the heavy chain-coding region of an IgG1 subclass monoclonal antibody (pspHCIgG1) which recognizes mammalian Tissue Factor protein. Plasmid ICP 1221 (FIG. 7) contains a kappa light chain coding region (pspLCIgG1/4) that together with the above mentioned heavy chain forms a full chain monoclonal antibody with desired specificity. In both clones, standard methods were used to generate restriction ends to facilitate cloning. Both coding regions are liberated as NcoI to XbaI restriction fragments. In the example shown in (FIG. 8) the light chain region was cloned into a plant expression vector adjacent to the (OCS)3MAS promoter and subsequently the IRES (cp148) and heavy chain were inserted 3′ to that and followed by a Nos transcription termination signal. The same vector carries a plant selectable marker (BAR) under the transcriptional control of the 2×35S promoter (pICGHpolyAb1, FIG. 8).

The DNA construct thus resembles the molecule described in FIG. 1 whereby the light chain gene is Gene 1 and the heavy chain gene is Gene 2. A similar plasmid was constructed in which the order of the heavy and light chain genes are reversed. This vector was subsequently transferred into Agrobacterium and used for transient expression and transformation of Arabidopsis thaliana, N. benthamiana, Brassica juncea and B. campestris. Agrobacterium transformation of Arabidopsis was carried out using the vacuum infiltration method although it is recognized that there are numerous protocols for performing Agrobacterium mediated plant transformation. Transient expression assays were performed using vacuum infiltration of leaf explants and whole seedlings.

In the example shown in FIG. 9, the structural gene encodes the light chain of an antibody. The gene is cloned into a plant expression vector adjacent to the (OCS)3MAS promoter and as shown in the Figure, the IRES (cp148) and the plant selectable marker (NPTII) are inserted 3′ to the structural gene. A CaMV 35S transcription termination signal is provided at the 3′-end of this construct. The same vector carries a gene encoding the heavy chain of the antibody cloned adjacent to the (OCS)3MAS promoter. The IRES (cp148) and the plant selectable marker (BAR) are inserted 3′ to the heavy chain gene and are followed by a CaMV 35S transcription termination signal (pXB1500, FIG. 9). In this fashion, the DNA construct resembles the molecule described in FIG. 1 whereby an antibody chain gene is Gene 1 and the selectable marker gene is Gene 2.

A similar plasmid was constructed in which the order of the heavy and light chain genes was reversed. This vector can be subsequently transferred into Agrobacterium and used for transient expression and transformation of Arabidopsis thaliana, N. benthamiana, Brassica juncea and B. campestris as described above. Agrobacterium transformation of Arabidopsis can be carried out using the vacuum infiltration method although, as it is recognized that there are numerous protocols for performing Agrobacterium-mediated plant transformation. Transient expression assays can be performed using vacuum infiltration of leaf explants and whole seedlings as is known in the art.

In the case of the Agrobacterium transformation, the T1 seed was germinated on media containing the selectable agent and survivors were then screened by PCR analysis for the presence of the heavy and light chain coding regions. Materials testing positive in this manner were further propagated and tested by western blot analysis and ELISA.

### Example 2

. In this example the production of a monoclonal antibody is described.

Plasmid ICP1177 (FIG. 9) includes the heavy chain-coding region of an IgG4 subclass monoclonal antibody (pspHCIgG4). Plasmid ICP1221 (FIG. 7) contains a kappa light chain-coding region (pspLCIgG1/4) that together with the above mentioned heavy chain forms a full chain monoclonal antibody with desired specificity.

The cloning procedures (yielding pICGHpolyAb4, FIG. 10), plant transformation and selection as well as the analysis of the product were essentially as described in Example 1.

### Example 3

Example 3. In this example, there are three coding regions being driven by a single promoter. In this case the plant selectable marker has been included directly into the DNA construct as the 5′-most gene adjacent to the promoter and the heavy chain is inserted downstream of that with the cp148 IRES at its 5′ end. The light chain gene is inserted downstream of that having the mp75 IRES at it's 5′ end and then lastly a termination/polyA site. An alternative configuration places polycistronic heavy and light chain gene driven by a promoter as in Examples 1 and 2 and the selectable marker with its own promoter on the same DNA construct. In this fashion the antibody genes are placed under the control of one type of promoter and the selectable gene on another. This provides tighter linkage of the marker and the antibody genes compared to the co-transformation methods described in examples 1 and 2 but still allows for separate and distinct regulation of the expression of the genes.

All patent and non-patent publications cited in this specification are indicative of the level of skill of those skilled in the art to which this invention pertains. All these publications and patent applications are herein incorporated by reference to the same extent as if each individual publication or patent application was specifically and individually indicated as being incorporated by reference herein.

Those skilled in the art will recognize, or be able to ascertain, using no more than routine experimentation, numerous equivalents to the specific substances and procedures described herein. Such equivalents are considered to be within the scope of this invention, and are covered by the following claims.

What is claimed is:

1. A nucleic acid construct, comprising the following elements functional in a plant cell and operably linked from 5′ to 3′; a transcriptional regulatory element, a first coding region encoding a first polypeptide comprising a first portion of an immunologically active portion of an antibody capable of specifically binding to an antigen, an IRES element, a second coding region encoding a second polypeptide comprising a second portion of the immunologically active portion of the antibody capable of specifically binding to an antigen, wherein when said first and second portions are expressed, they associate to form a multi-subunit polypeptide capable of specifically binding to the antigen.

2. A nucleic acid construct, comprising the following elements functional in a plant cell and operably linked from 5′ to 3′, a transcriptional regulatory element, a first coding region encoding a first polypeptide subunit of a multi-subunit protein, an IRES element, and a second coding region encoding a second polypeptide subunit of a multi-subunit protein, wherein said first and second coding regions do not encode the same subunit.

3. A nucleic acid construct, the following elements functional in a plant cell and operably linked a transcriptional regulatory element, at least one first coding region encoding a processing protein for processing an immature protein to a mature protein, an IRES element functional in the plant cell, and a second coding region encoding the immature protein, wherein expression of the first and second coding region in the same plant cell results in processing of the immature protein to its mature form, the IRES element is between coding regions, and the transcriptional regulatory element transcribes a polycistronic transcript encoding both the first and second coding region.

4. An nucleic acid construct for expressing an exogenous multi-subunit polypeptide in a host plant cell, comprising a sequence encoding a polycistronic mRNA encoding a exogenous multi-subunit protein, wherein the exogenous polypeptide is not naturally expressed in the host plant cell.

5. An nucleic acid construct for expressing a polypeptide in a plant cell comprising a sequence encoding a polycistronic mRNA encoding a single chain T Cell Receptor, single chain MHC molecule, a single chain protein of the immunoglobulin superfamily or fusions thereof.

6. The nucleic acid construct of claim 1, wherein the first coding region and second coding region encode a heavy or light chain of the antibody and wherein the first and second coding regions do not encode the same chain.

7. The nucleic acid construct of any of claims 1-5, further comprising a termination signal.

8. The nucleic acid construct of any of claims 1-5, wherein the first and second coding regions further comprise a targeting sequence.

9. The nucleic acid construct of any of claims 1-5, wherein the transcriptional regulatory element is a promoter.

10. The nucleic acid construct of any of claims 1-5, wherein the transcriptional regulatory element is replaced with an IRES element functional in the plant cell and the genomic locus of integration provides the transcriptional control of the engineered construct.

11. The nucleic acid construct of claim 1, wherein the antibody is a monoclonal antibody.

12. The nucleic acid construct of any of claims 1-5, wherein the IRES element is IRESmp75.

13. The nucleic acid construct of any of claims 1-5, wherein said IRES element is IREScp148.

14. The nucleic acid construct of any of claims 1-5, wherein the targeting sequence targets polypeptide products of the first and second coding regions to the endoplasmic reticulum of the plant cell.

15. The nucleic acid construct of claim 8, wherein the targeting sequence is a transit peptide that targets the polypeptide products of the first and second coding regions to a plastid of the plant cell.

16. The nucleic acid construct of claim 15, wherein the plastid is a chloroplast.

17. The nucleic acid construct of any of claim 8, wherein the targeting sequence is a transit peptide that targets the polypeptide products of the first and second coding regions to a mitochondrion of the plant cell.

18. The nucleic acid construct of claim 1,wherein the first coding region encodes the heavy chain of the antibody molecule and said second coding region encodes the light chain of the antibody molecule.

19. The nucleic acid construct of claim 1, wherein said first coding region encodes the light chain of the antibody molecule and said second coding region encodes the heavy chain of the antibody molecule.

20. The nucleic acid construct of claim 1, wherein the antibody is human or humanized.

21. The nucleic acid construct of any of claims 1-5, further comprising a gene encoding a selectable marker.

22. The nucleic acid construct according to claim 21, wherein the gene encoding the selectable marker is operably linked to a promoter that drives the expression of the marker.

23. The nucleic acid construct of any of claims 1-5, further comprising at least one eukaryotic origin of replication.

24. The nucleic acid construct of any of claims 1-5, further comprising a prokaryotic origin of replication.

25. The nucleic acid construct of claim 23, further comprising a prokaryotic origin of replication.

26. The nucleic acid construct of any of claims 1-5, further comprising one or more additional structural genes comprising an IRES element 5′ to the one or more additional structural genes.

27. The nucleic acid construct of claim 3, wherein the immature protein is preproinsulin.

28. The nucleic acid construct of claim 8, wherein targeting is to an apoplast, vacuole, chloroplast, plastid, mitochondria, peroxisome or nucleus, or to the cell wall.

29. A composition comprising a first expression unit and a second expression unit, wherein the first expression unit comprises the nucleic acid construct according to any of claims 1-5, and the second expression unit comprises a third coding region operably linked to a promoter or IRES element.

30. A plant or portion thereof comprising the nucleic acid construct of any of claims 1-5.

31. The plant or portion thereof of claim 30, wherein the plant is selected from the group consisting of Arabidopsis, Brassica, maize, alfalfa, soybean, tobacco, crucifera, cottonseed, sunflower, and legumes.

32. A method for producing a host plant cell capable of expressing an exogenous protein not naturally produced in the plant cell, comprising: introducing the nucleic acid construct of any of claims 1-5, into the host plant cell.

33. The method of claim 32, further comprising propagating a plant from the plant cell.

34. The method of claim 33, further comprising cultivating the progeny of the plant.

35. The method of claim 32, wherein the plant cell is from a tissue selected from the group consisting of protoplast, cells, callus tissue, suspension culture, leaf, roots, stem, hypocotyls, pollen, seed, and meristem.

36. The method of claim 32, further comprising the step of expressing the protein.

37. The method of claim 32, wherein the protein is selected from the group consisting of: an antibody, T cell receptor, an MHC protein, a protein of the immunoglobulin superfamily, interferon, interleukin, hormone, an antigen, a receptor, and a therapeutic protein.

38. The method of claim 32, wherein the protein is a fusion protein.

39. The method of claim 38, wherein the fusion protein comprises an effector molecule.

40. A host plant or portion thereof comprising at least one cell comprising a nucleic acid encoding a polycistronic mRNA encoding a exogenous multi-subunit protein, the exogenous protein being one not naturally expressed in the host plant.

41. The plant or portion thereof of claim 40, wherein the plant is an F₀ plant.

42. The plant or portion thereof of claim 40, wherein the plant is Arabidopsis.

43. The plant or portion thereof according to any of claims 40-42, wherein the multi-subunit protein comprises a heterodimeric or heteromultimeric protein selected from the group consisting of a T Cell Receptor, MHC molecule, protein of the immunoglobulin superfamily or co-receptors, nucleic acid binding protein, abzyme, receptor, growth factor, cell membrane protein, differentiation factor, hemoglobin like protein, and a multimeric kinase.

44. A plant or portion thereof comprising at least one cell comprising a nucleic acid encoding a polycistronic mRNA encoding an inactive polypeptide which is capable of being modified to an active form and a processing protein for processing the inactive protein to the active form.

45. The plant or portion thereof according to claim 44 wherein the processing protein is a protease.

46. The plant or portion thereof according to any of claims 44-45, wherein the inactive protein is preproinsulin.

47. The plant or portion thereof of claim 44, wherein the processing protein is an enzyme for adding a modification to the protein.

48. The plant or portion thereof of claim 47, wherein the enzyme is a kinase.

49. A method for producing a host plant cell capable of expressing an exogenous multi-subunit protein not naturally expressed in a host plant cell, comprising:
expressing a nucleic acid encoding a polycistronic mRNA encoding the multi-subunit protein in the plant cell.

50. The method according to claim 49, wherein the plant cell is from an F₀ plant.

51. The method according to claim 49, wherein the plant cell is an Arabadopsis cell.

52. The method according to any of claims 49-51, wherein the multi-subunit protein comprises a heterodimeric or heteromultimeric protein selected from the group consisting of a T Cell Receptor, MHC molecule, protein of the immunoglobulin superfamily or co-receptors, nucleic acid binding protein, abzymes, receptor, growth factor, cell membrane protein, differentiation factor, hemoglobin like protein, and a multimeric kinase.

53. A method for producing an active form of an exogenous protein in a plant comprising expressing a nucleic acid encoding a polycistronic mRNA encoding an inactive polypeptide which is capable of being modified to an active form and a processing protein for processing the inactive protein to the active form.

54. The method of claim 53, wherein the processing protein is a protease.

55. The method of claim 53 or 54, wherein the inactive protein is preproinsulin.

56. The method of claim 52, wherein the processing protein is an enzyme for adding a modification to the protein.

57. The method of claim 56, wherein the enzyme is a kinase.
